(19) United States
(12) Patent Application Publication
(10) Pub. No.: US 2013/0213405 A1
(43) Pub. Date: Aug. 22, 2013

(54) Method and apparatus for releasably holding a ventilator tube to a tracheostomy tube

(71) Applicant: Iris Murray Dillworth, Clemmons, NC (US)

(72) Inventor: Iris Murray Dillworth, Clemmons, NC (US)

(21) Appl. No.: 13/385,385

(22) Filed: Feb. 16, 2012

(51) Int. Cl. A61M 16/04

(52) U.S. Cl. 128/207.17

(57) ABSTRACT

A ventilator tube holder is provided in the form of a soft patch having opposed ends which are Velcroed to underlying tracheostomy ties, with a central portion having a circular orifice through which the ventilator tube passes and is held. In one embodiment the circular orifice is provided with a notch or slit for easy mounting of the patch around the ventilator tube or for quick removal of the ventilator tube, whereas in a further embodiment a soft elastic tie or strip is Velcroed from one side of the orifice to the other to hold the ventilator tube in place or to tighten the ventilator tube to the patch during any kind of deformation or stretching that may occur.

### FIELD OF THE INVENTION

This invention relates to a tracheostomy ventilator tubes and more particularly to a retention device for holding the ventilator tube to the tracheostomy tube in a secure but releasable fashion.

### BACKGROUND OF THE INVENTION

As is well known ventilator tubes, called vent tubes, are attachable to a tracheostomy tube or trach tube in which the ventilator tube is pressed fit to the tracheostomy tube.

In pediatric tracheostomy often times the ventilator tube becomes disassembled or falls off the tracheostomy tube and when this happens an alarm sounds and the ventilator tube must be quickly reattached to the tracheostomy tube to continue the air flow to the patient.

The problem of the detachment of the ventilator tube to the tracheostomy tube is particularly severe in pediatric patients. This is because young patients are restless, move around and this gives rise to detachment of the ventilator tube.

It is noted that the ventilator tube is utilized to pump air into the patient's lungs and to evacuate the patient's lungs on a periodic basis. Also the ventilator tubing is sometimes attached to a humidifier.

In short, the ventilator tubing is attached to the tracheostomy tube to help a child to breath. It is a very common occurrence that the ventilator tube keeps disconnecting from the tracheostomy tube, due to patient movement as well as when the patient must be suctioned.

While there have been attempts to clip the ventilator tube to the tracheostomy tube as shown in U.S. Pat. No. 6,588,426, such attachment methods involving detent clips for preventing unwanted separation of the tubes are difficult to utilize due, to the stiff nature of the attachment device.

In the prior art a so-called Strong Man attachment device is used in an attempt to keep the ventilator tube from falling off the tracheostomy tube. In this device two straps run between separated clips, with each clip attached to an associated tracheostomy tie. The problems with such device are first and foremost that the straps utilized to surround the ventilator tube are stiff and irritates the patient's neck. The second problem is that often times it is necessary to quickly detach the ventilator tube from the tracheostomy tube to suction the patient. In order to do so with the Strong Man type device the clips must be unclipped from their tracheostomy ties. This is often times difficult due to the fact that the clips involve separating the opposed ends of the u-shaped clip so that the clip may be slipped out from around the tracheostomy tie.

As a result, both due to the irritation and the inability to quickly release the Strong Man system, this device is largely in disuse. Moreover, the two straps that are supposed to clamp the body of the ventilator tube to the tracheostomy tube become separated and the ventilator tube separates from the tracheostomy tube, regardless of strap tension.

In an effort to solve the irritation and detachment problem associated with the Strong Man tracheostomy appliance, Iris Dillworth, the inventor of the subject invention invented a new method for securing the ventilator or tube to the tracheostomy tube. In order to do this the Dillworth system used a foam patch having arms at either end and a large center portion, with the center portion provided with a slit through which the ventilator tube passes. The arms of the patch are provided with Velcro patches to attach them to respective tracheostomy tube ties for securing the ventilator tube to the tracheostomy tube. The patch is soft in construction, having a central foam portion with soft backing and facing material.

In order to hold the ventilator tube to the tracheostomy tube the slit engages the ventilator tube to hold the ventilator tube securely onto the tracheostomy tube.

However, as is often the case, it is necessary to disconnect the ventilator tube to suction the patient. This may be done as many as fifty times in an eight hour time period and requires that one end of the patch be pulled off its tracheostomy tie. With the ventilator tube removed to one side it is possible to suction the patient as well as to clean around the tracheostomy tube, often times with a Q-tip. As the tracheostomy tube is usually covered with secretions. A buildup of secretions can cause inflammation at the tracheostomy site leading to infection. It is therefore important to be able to clean around the tracheostomy tube site in a convenient manner.

Most importantly however, with constant patient motion the ventilator tube moves and comes off of the tracheostomy tube because the slit stretches and widens with usage. Thus, when the slit stretches and the ventilator tube can slip and can become disengaged from the tracheostomy tube.

Note that in practice the useful lifetime of such a slit patch is oftentimes no more that ten removal and attachment cycles, for instance for suctioning, requiring another solution to the secure holding of the ventilator tube to the tracheostomy tube.

### SUMMARY OF THE INVENTION

Rather than utilizing the Strong Man straps or the slitted patch, in the subject invention the patch is provided with a circular orifice within the enlarged portion of the patch in which the diameter is made coincident with the diameter of the ventilator tube. In one embodiment the top portion of the orifice is slitted or notched so that the ventilator tube can be easily slipped from the circular orifice, when for instance removal for suctioning is required. This means that the ventilator tube can be removed without detaching the patch as was required with the slitted patch.

Note, the circular orifice does not stretch to any great degree, unlike the slit and is therefore much more secure than the slitted patch.

Moreover, in order to more securely attach the ventilator tube to the tracheostomy tube a cotton elastic strap is used to hold the ventilator tube to the patch. In this case the cotton strap is made to overlie the ventilator tube to keep it in place, with the cotton strap being Velcroed from one side of the circular orifice to the other utilizing Velcro pads on the patch to either side of the orifice. The elastic strap in one embodiment is a cotton strap which does not irritate the child's neck or throat area such that rather than irritating the child's neck as is the case with the Strong Man system, the soft cotton strap is merely there to hold the ventilator tube in place against patient movement.

The subject elongated patch with a circular orifice serves two purposes. The first purpose is to more securely attach the ventilator tube to the tracheostomy tube and is especially useful to prevent stretching due to the circular configuration.

Secondly, the utilization of a securing strap across the ventilator tube from one side of the orifice to the other is convenient both to tighten up the connection to the patch and also to permit quick removal of the ventilator tube from the tracheostomy tube for instance, to suction the patient without having to unhitch one end of the patch.

With the slitted patch, the patch had to be swung away to remove the ventilator tube from the tracheostomy tube since the tracheostomy tube could not come out of the slit. In one embodiment the patch may be kept in place and the ventilator tube removed simply by undoing the strap and removing the tube through the upper slit in the circular orifice.

Additionally, removal of the ventilator tube may be accomplished by detaching one of the ends of the patch from its associated tracheostomy tie which in one embodiment is a Velcro type attachment such that the ventilator tube and patch may be swung away from the tracheostomy tube to permit suctioning of patients.

Due to the requirement of frequent suctioning a patient this quick disconnect is an important factor in the design of the subject ventilator tube holder.

Moreover, because the strap which holds the ventilator tube in the circular orifice may be quickly removed from one side or the other, the tracheostomy tube area can be cleaned with a Q-tip, without removing the ventilator tube. Additionally, since the ventilator tube can be removed quite quickly by removing the strap and slipping the ventilator tube out of the orifice slit, cleaning is quickly achieved because the areas of secretion around the tracheostomy tube are exposed with the removal of the ventilator tube.

In one embodiment, the ends of the subject patch are shorter for instance than the lengths of the tracheostomy ties which are themselves Velcroed to a collar around the patient.

What this means is that the tracheostomy tube may be removed simply by removing one of the ends of the patch from its associated tracheostomy tie as by simply ripping off the Velcro and swinging the patch and ventilator tube away from the tracheostomy tube. Secondly, in an emergency operation the tracheostomy tie as well as the patch it carries may be stripped away from the tracheostomy collar for quick removal of the apparatus.

As can be seen, because of the difference in the length of the end of the patch versus the end of the tracheostomy tie, a number of different functions can be achieved. First the patch may be swung away from the tracheostomy tube by merely unVelcroing the end from the tracheostomy tie. Secondly, the entire apparatus including tracheostomy tube and ventilator tube may be removed in an emergency simply by ripping off the tracheostomy tie which carries with it the patch and the ventilator tube.

While the subject holder has been described for pediatric patients, it is likewise applicable to adult use, with the size of the patch elongated for adult use.

In summary, a ventilator tube holder is provided in the form of a soft patch having opposed ends which are Velcroed to underlying tracheostomy ties with a central portion having a circular orifice through which the ventilator tube passes and is held. In one embodiment the circular orifice is provided with a notch or slit for easy mounting of the patch around the ventilator tube or for quick removal of the ventilator tube, whereas in a further embodiment a soft elastic tie or strip is Velcroed from one side of the orifice to the other to hold the ventilator tube in place or to tighten the ventilator to the patch during any kind of deformation or stretching that may occur.

### BRIEF DESCRIPTION OF THE DRAWINGS

These and other features of the subject invention will be better understood in connection with the Detailed Description, in conjunction with the Drawings, of which:

FIG. 1 is a diagrammatic illustration of the attachment of a ventilator tube to a tracheostomy tube which is held in place by tracheostomy ties that are Velcroed to a collar around the neck of a patient;

FIG. 2 is a diagrammatic illustration of a disconnection between the ventilator tube and the tracheostomy tube which often times is fatal and which disconnection is often alarmed so as to let the personnel know to quickly reattach the ventilator tube;

FIG. 3 is a diagrammatic illustration of the Strong Man type holder for a ventilator tube, illustrating strap-holding clips that are clipped to the tracheostomy tube ties, also illustrating an area of irritation on a patient's neck above the tracheostomy tube due to the stiff nature of the Strong Man straps;

FIG. 4A is a diagrammatic illustration of the Strong Man tie illustrating a strap loop secured using clips to standard tracheostomy ties;

FIG. 4B is a diagrammatic illustration of the use of the Strong Man holder of FIG. 4 illustrating the securing of a ventilator tube to a tracheostomy tube through the Velcroing of one end of the Strong Man loop through the associated clip;

FIG. 4C is a diagrammatic illustration of the utilizing of the Strong Man holder to hold a ventilator tube in place, illustrating the looped strap of the Strong Man holder, as well as the clips that secure the loop to the tracheostomy tube ties;

FIG. 5A is a diagrammatic illustration of a prior art holder involving a patch having a foam sandwiched between facings and moving opposed ends that are Velcroed to positions on underlying tracheostomy ties, showing a slit in the large central portion of the patch;

FIG. 5B is a diagrammatic illustration of the patch of FIG. 5A, illustrating that the slit in the holder in FIG. 5A is utilized to secure the ventilator tube to the tracheostomy tube, also showing one of the ends of the patch that is to be Velcroed to an underlying tracheostomy tube tie;

FIG. 5C is a diagrammatic illustration of the patches of FIG. 5A and FIG. 5B, illustrating the stretching of the slit due to patient movement and the necessity of frequent suctioning resulting in dislodgement of the ventilator tube;

FIG. 6A is a diagrammatic illustration of the subject holder showing a patch having Velcroable ends, a large central portion and a circular orifice or hole in the central portion for securing a ventilator tube, with the orifice having a notched or slitted top so that a ventilator tube may be easily installed and removed, also showing a soft elastic cotton strap overlying an upper portion of the circular orifice for holding the ventilator tube in place;

FIG. 6B is a diagrammatic illustration of the patch of FIG. 6A showing the circular orifice, the slit and the elastic strap;

FIG. 7 is a diagrammatic illustration showing the removal of the subject patch by removing one of the ends of the patch from the underlying tracheostomy tie;

FIG. 8 is a diagrammatic illustration of the subject patch in place in which the ventilator tube is held securely to a tracheostomy tube with a soft elastic strap overlying the top portion of the ventilator tube, the elastic strap having been Velcroed at one end to a Velcro patch on the pad and showing it Velcroed at the other end to another Velcro pad; and,

FIG. 9 is a diagrammatic illustration of two methods of removing the ventilator tube from the tracheostomy tube in an emergency showing either the removal of the patch by the lifting of one of its ends from a tracheostomy tie, or by removing the tracheostomy tie itself from the tracheostomy collar which also results in the removal of the patch as it is swung away.

### DETAILED DESCRIPTION

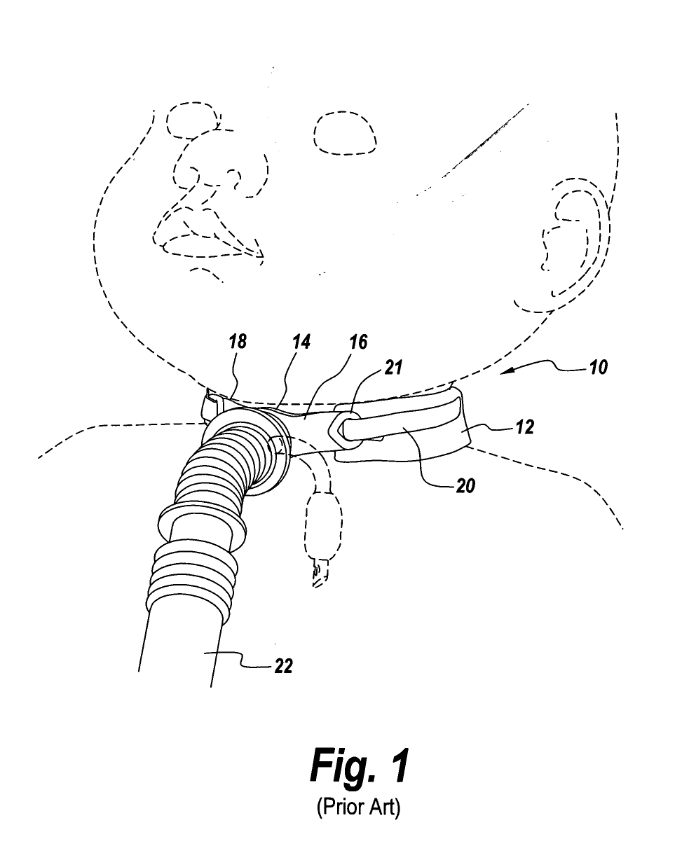

Referring now to FIG. 1 a patient 10 is provided with a tracheostomy collar 12 to which is secured a tracheostomy tube 14 that is held in place by a holder having ends 16 and 18 secured to collar 12 utilizing tracheostomy ties 20 that pass through rings 21 and the ends 16 and 18 of the tracheostomy tube holder. Note these ties are Velcroed to themselves and to collar 12 to hold the tracheostomy tube in place.

The tracheostomy tube 14 is provided with a ventilator tube 22 which is held in place in a friction fit.

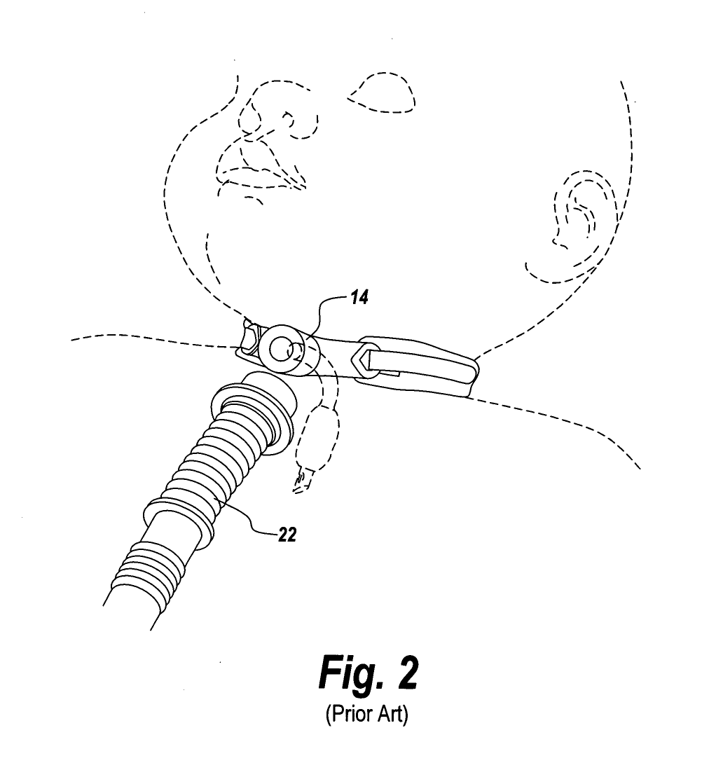

Referring to FIG. 2, tracheostomy tube 14 is shown exposed, with ventilator tube 22 becoming dislodged or removed. This causes a serious often life-threatening situation because the patient can no longer receive air.

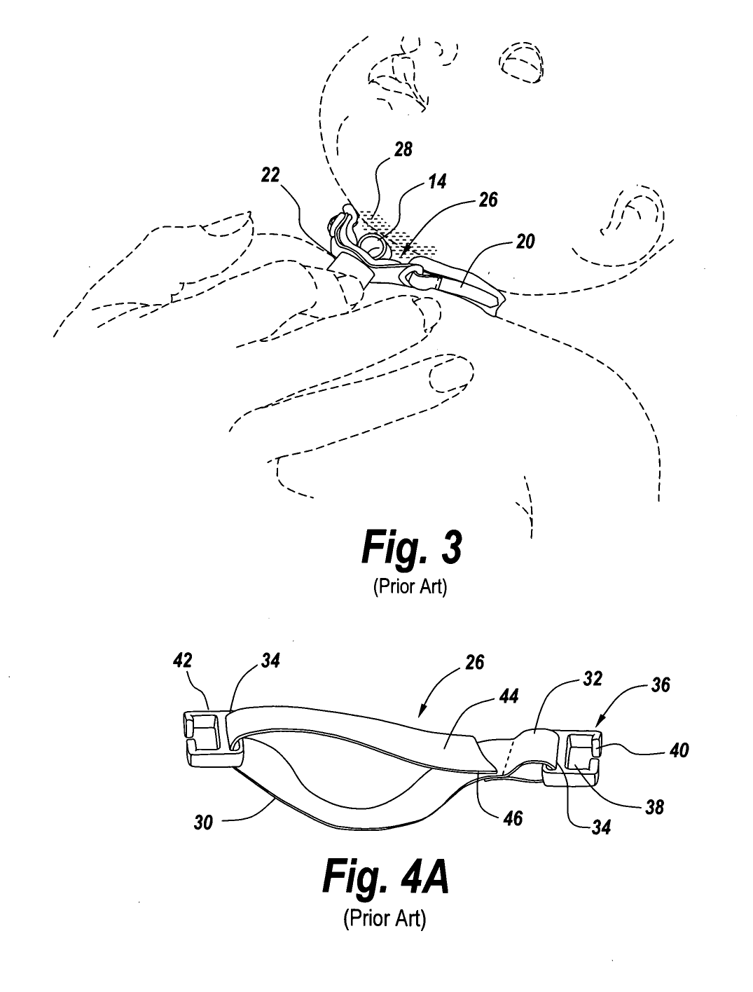

Referring to FIG. 3, in the prior art in order to keep ventilator tube 22 in place on tracheostomy tube 14 a so-called Strong Man device 26 is utilized to grip the ventilator tube and hold it securely to the tracheostomy tube using a strap loop.

Prior to describing the strap loop it will be noted that because the strap structure is relatively stiff there is an area 28 on the neck of the patient which is abraded by the strap, with the strap causing irritation, inflammation and sometimes infection.

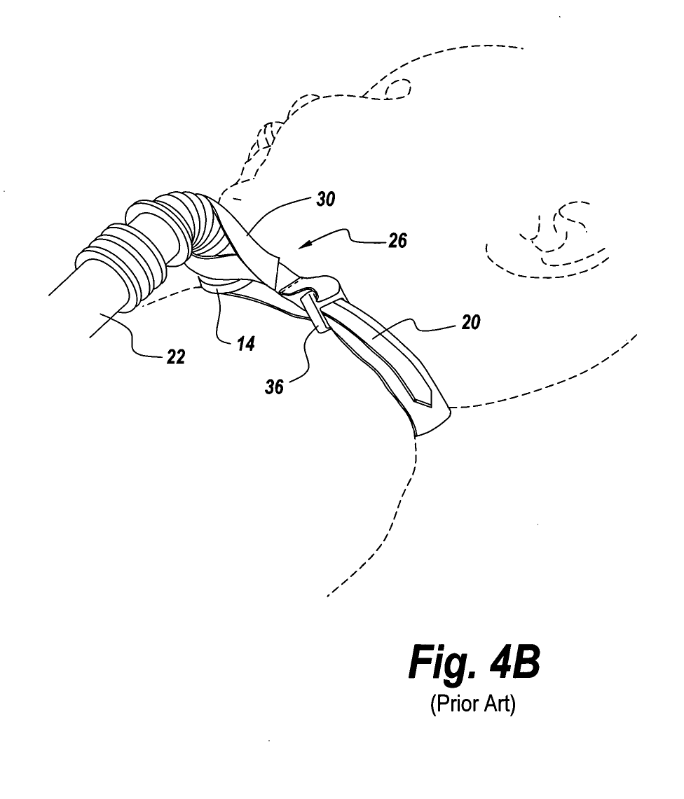

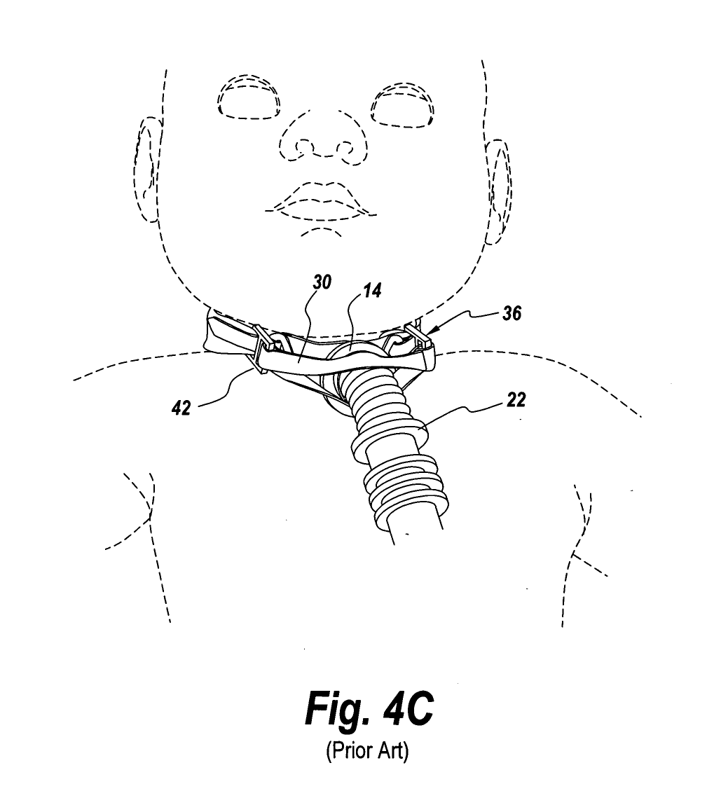

Referring to FIG. 4A, the Strong Man device 26 is shown to include a strap loop 30 which has one end 32 looped through a slot 34 in a clip 36 that has a u-shaped orifice 38 and a slot 40 so that this clip may be clipped around the tracheostomy tie 20 in FIG. 3.

Also shown in FIG. 4A is an opposed clip 42 of a similar configuration to that of clip 36. Here a slot 34 encircles one end of loop 30, with the free end 44 of loop 30 being Velcroed to itself as illustrated at 46.

As illustrated in FIG. 4B, Strong Man holder device 26 is shown in place with clip 36 surrounding tracheostomy tie 20. Here loop 30 is clearly shown holding the ventilator tube 22 to the tracheostomy tube 14.

FIG. 4C shows a front view of the attachment of ventilator tube 22 to tracheostomy tube 14, clearly showing clips 36 and 42 as well as loop 30 that surrounds ventilator tube 22.

From the drawings it will be appreciated that the ability of this holder to securely hold the ventilator tube to the tracheostomy tube is in question because it requires a significant amount of loop tension on loop 30 to press against the exterior surface of the ventilator tube. Moreover, the ability to hold the ventilator tube to the tracheostomy tube is in question because the holding ability of this device is dependent upon the inelasticity of loop 30.

Also, since the clips are spaced apart and not close to the ventilator tube, a large amount of tension must be placed on loop 30 in order to properly secure the ventilator tube to the tracheostomy tube, noting the large amount of tension required due makes the loop stiffer. As mentioned above, the stiffness of the loop causes irritation to the neck of the patient.

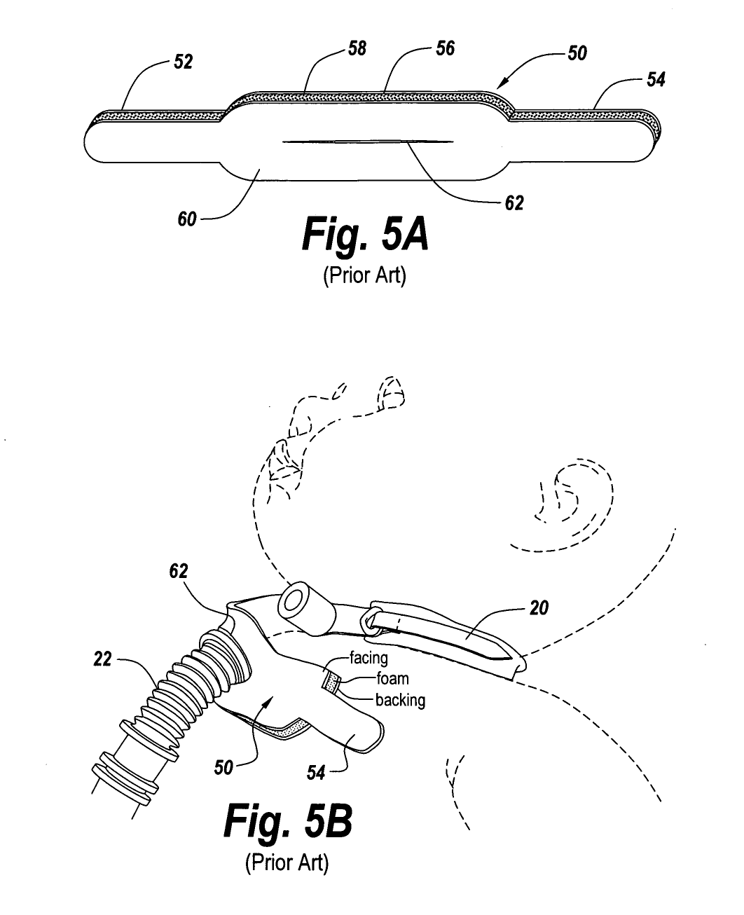

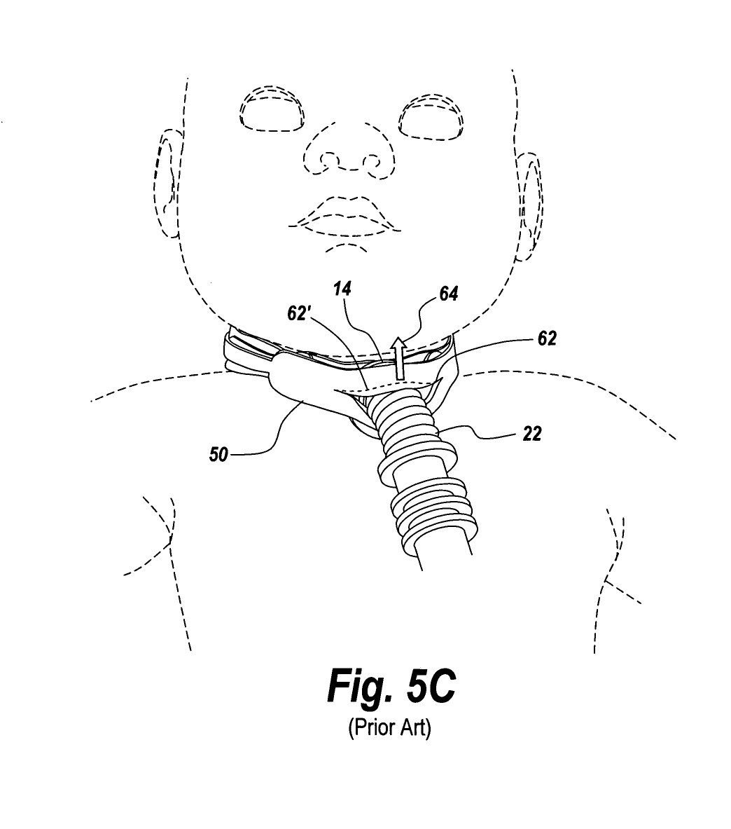

Referring now to FIG. 5A, a prior art patch 50 is used as a holder for a ventilator tube in which the patch has opposed ends 52 and 54 that are Velcroed to the underlying tracheostomy ties.

In one embodiment, the patch is made of a foam core 56 which is sandwiched between a flexible backing member 58 and a flexible facing member 60.

Patch 50 is provided with a slit 62 which as illustrated in FIG. 5B is expanded to accommodate ventilator tube 22 in a tight fit. When the ends 54 of patch 50 are secured to tracheostomy tie 20 for instance by Velcroing the ends to an underlying tracheostomy tie, as illustrated in FIG. 5C patch 50 secures ventilator tube 22 to tracheostomy tube 14.

The problem with such a patch is that during usage slit 62 becomes stretched as illustrated at 62¹ and by arrow 64. As mentioned, this causes the ventilator tube to slip away from the tracheostomy tube which is a defect in this type of patch.

In order to solve the problem of the stretching of the slit 62 and to provide a secure holder for a ventilator tube, a patch 70 is constructed with ends 72 and 74 having Velcro pads shown in dotted outline respectively at 76 and 78.

Patch 70 has an expanded portion 80 provided with a circular orifice 82 which in one embodiment has a slit or notch 84 rising vertically as shown.

Patch 70 also in one embodiment has a foam core 86 faced with a flexible facing 88 and backed with a flexible backing 90.

In order to further retain the ventilator tube in orifice 82 a cotton elastic strap 92 is Velcroed at either end to Velcro pads 94 and 96 on facing 88, the purpose of which is to retain the ventilator tube within orifice 82 and to be able to tighten this connection to counter the natural stretching of the patch during usage.

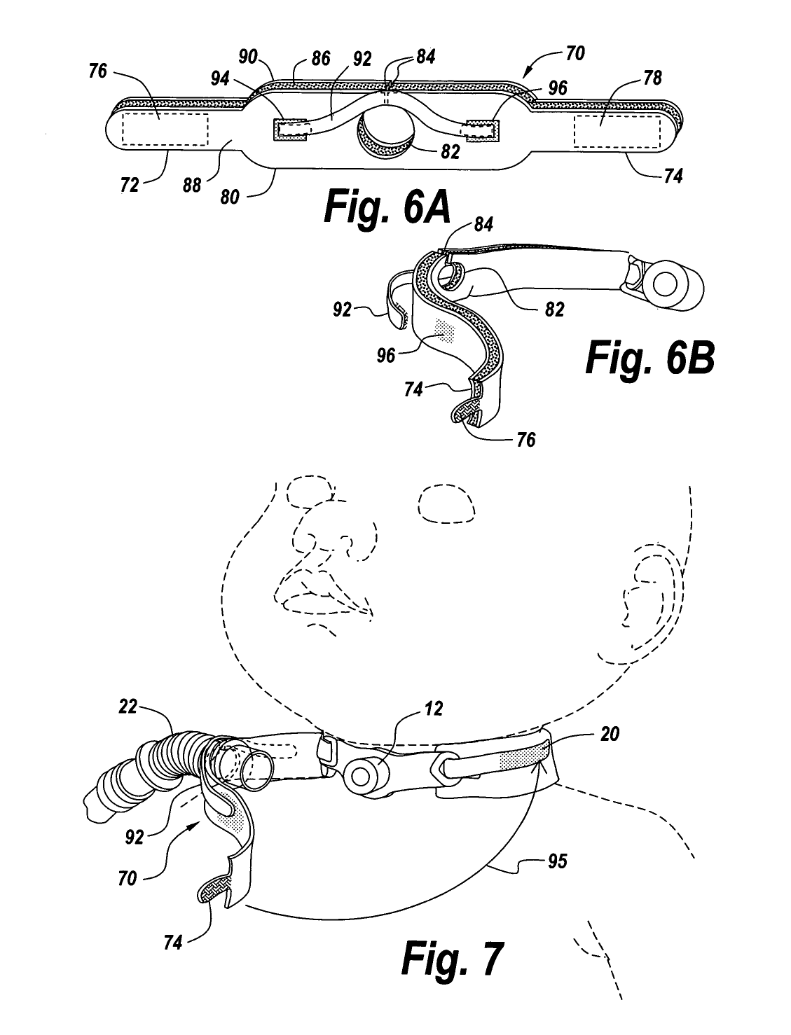

Referring to FIG. 6B, it can be seen is that end 74 is provided with a Velcro pad 76, with the slitting 84 of orifice 82 being clearly visible. Also elastic cotton strap 92 is shown having one end to be Velcroed to Velcro pad 96.

Referring to FIG. 7, in an operation, ventilator tube 22 is secured to tracheostomy tube 12 by moving patch 70 and its corresponding ventilator tube onto the tracheostomy tube, whereupon end 74 is Velcroed to tracheostomy tie 20 as illustrated by arrow 95.

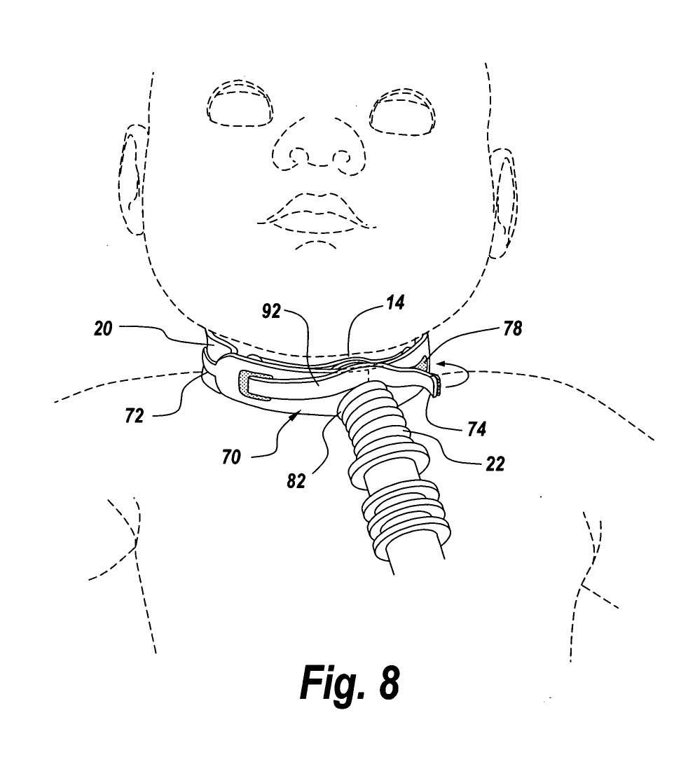

Referring to FIG. 8, what is seen that patch 70 securely mounts ventilator tube 22 to tracheostomy tube 14 through the Velcro fastening of for instance end 72 to the underlying tracheostomy tie 20. Here it can be seen that orifice 82 tightly surrounds ventilator tube 22 and with the securing of patch 72 at either end to the tracheostomy ties the ventilator tube is securely fastened to the tracheostomy tube. Moreover, as can be seen, cotton elastic strap 92 is Velcroed at end 74 to the Velcro pad 78 on patch 70.

It is noted that due to the softness of the elastic strip as well as the softness of the patch itself very little if any irritation of the patient occurs.

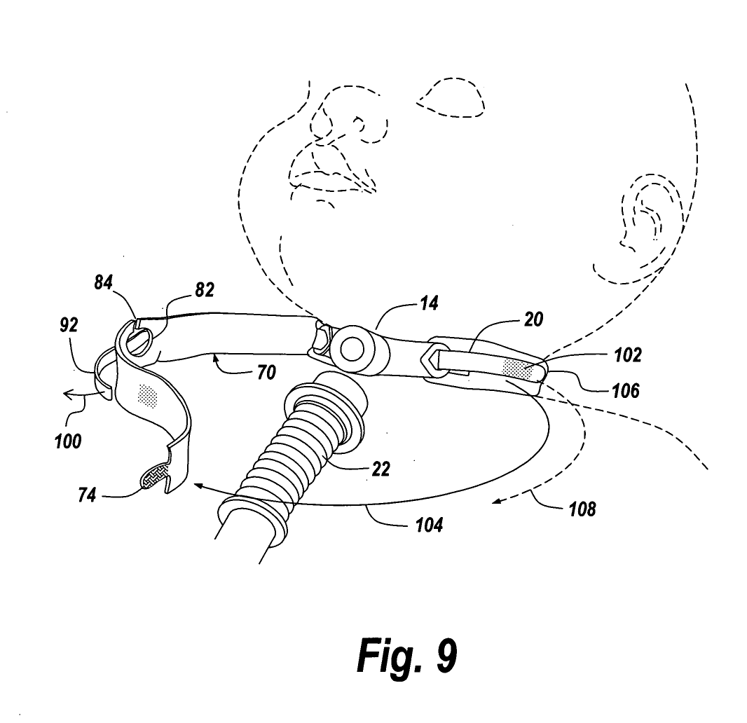

Referring to FIG. 9, when ventilator tube 22 is to be removed from tracheostomy tube 14 a quick release is achieved either through removal of elastic strap 92 as illustrated by 100 or by the removal of patch 70 by the stripping of distal end 74 from the Velcro attachment point 102 to tracheostomy tie 20 as illustrated by arrow 104. A third way of emergency removal is to remove the tracheostomy tube itself by pulling off the end 106 of tracheostomy tie 20 as illustrated by dotted arrow 108.

Thus, the subject system allows for emergency removal of the ventilator tube either through slipping it out of orifice 82 via slot 84 by removal of strip 92; or by removal of patch 70; or finally by the removal of the tracheostomy tube itself along with all connected apparatus.

While the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

What is claimed is:

1. A ventilator tube holder for use with underlying tracheostomy ties, comprising:
a soft patch having opposed ends adapted to be attached to underlying tracheostomy ties, said soft patch having a central position including an orifice through which a ventilator tube is adapted to pass and be held, said patch being provided with a slit running from an edge thereof to said orifice.

2. The ventilator tube holder of claim 1, wherein said orifice is round and of a diameter to match the outside diameter of a ventilator tube, whereby said ventilator tube is adapted to be securely held by the interior surface of said round orifice.

3. The ventilator tube holder of claim 1, and further including a structure for closing said slit after a ventilator tube is inserted in the associated orifice.

4. The ventilator tube holder of claim 3, wherein said structure includes a strap releasably anchored to said patch at either side of said orifice.

5. The ventilator tube holder of claim 4, wherein said strap attachment to said patch on either side of said orifice utilizes a releasable loop attachment system.

6. The ventilator tube holder of claim 5, wherein said releasable loop attachment system includes a Velcro attachment system.

7. The ventilator tube holder of claim 4, wherein said strap includes a soft elastic tie.

8. The ventilator tube holder of claim 4, wherein said strap when in place overlies an inserted ventilator tube in said orifice and holds said ventilator tube in place.

9. The ventilator tube holder of claim 1, wherein said soft patch includes a foam core sandwiched between a backing and a facing member.

10. The ventilator tube holder of claim 9, wherein opposed ends of said backing are provided with a loop and hook attachment system.

11. The ventilator tube holder of claim 10, wherein said hook and loop attachment system includes a Velcro attachment system.

12. The ventilator tube holder of claim 1, wherein at least one of said opposed ends is releasably attachable to an underlying tracheostomy tie, whereby a tracheostomy tube held by said patch is quickly removable by the release of said releasably attachable end.

13. The ventilator tube holder of claim 1, wherein said tracheostomy tie is carried by a collar and wherein a ventilator tube held by said patch may be removed by unattaching the underlying tracheostomy tie from said collar.

14. The ventilator tube holder of claim 13, wherein said tracheostomy tie is releasably attached to a tracheostomy collar, whereby said ventilator tube may be removed by the removal of said underlying tracheostomy tie from said collar.

15. The ventilator tube holder of claim 1, wherein said patch is made of soft material so as not to irritate the neck of a patient when installed on said underlying tracheostomy ties.

16. The ventilator tube holder of claim 15, and further including a soft elastic tie releasably anchored at at least one end to said patch to one side of said orifice and anchored at the other end to said patch at a dramatically opposite position to the other side of said orifice, said soft elastic tie adapted to overlie a ventilator tube in said orifice to hold said ventilator tube in said circular orifice.

17. The ventilator tube holder of claim 16, wherein said soft elastic tie holds together the portions of said patch to either side of said slit.

18. The ventilator tube of claim 7, wherein said soft elastic tie includes cotton.

19. The ventilator tube of claim 18, wherein said cotton elastic tie has a looped surface at either end thereof and further including a hooked type structure in the form of a pad at at least one side of said orifice, said cotton and hook structure forming a releasable structure for said cotton to said pad.

20. A method of securing a ventilator tube to a patient wearing a collar to which are attached tracheostomy tubes, comprising the steps of:
securing the ventilator tube to the patient by placing a portion of the ventilator tube through an orifice in a soft patch having opposed arms; and
securing the arms to the tracheostomy ties.
